(19) United States
(12) Patent Application Publication
(10) Pub. No.: US 2007/0083249 A1
(43) Pub. Date: Apr. 12, 2007

(54) Healthful belt

(76) Inventor: Yu-Ting Tu, Tainan City (TW)

Correspondence Address: Yu-Ting Tu, 235 Chung-Ho, Box 8-24, Taipei, TW

(21) Appl. No.: 11/319,289

(22) Filed: Dec. 29, 2005

Related U.S. Application Data
(63) Continuation-in-part of application No. 11/246,054, filed on Oct. 11, 2005.

(51) Int. Cl. A61F 7/00

(52) U.S. Cl. 607/108; 607/112; 607/114

(57) ABSTRACT

A healthful belt includes an elastic cloth and a silica gel layer deposited on an inner surface of the elastic cloth. The silica gel layer is provided with plural air holes and far-infrared energy. Then the healthful belt is worn on a sore or painful section of a body, letting the far-infrared energy in the silica gel layer sink in the skin organization and the joint for functioning to give medicating effect to the sore and the pain, to reinforce blood circulation and metabolism, to relieve aching or sore, to get rid of fatigues, to activate metabolism, and helpful to the health of human bodies. In addition, a person may feel comfortable in using the healthful belt by the soft and ventilating layer of the silica gel directly contacting the skin of the user.

The present invention is a continuation in part (CIP) of the U.S. patent Ser. No. 11/246,054 assigned to the inventor and applicant of the present invention. Thus the content of U.S. patent Ser. No. 11/246,054 is incorporated into the present invention as a part of the present invention.

### BACKGROUND OF THE INVENTION

1. Field of the Invention

This invention relates to a healthful belt, particularly to one provided with a silica gel layer deposited on an inner surface of an elastic cloth and having plural air holes and far-infrared energy. When the healthful belt is worn a body section suffering pains, with the silica gel layer directly contacting the skin, the far-infrared energy sinks into the skin organization and the joint to promote blood circulation, getting rid of sourness and pains, strengthening metabolism, enhancing immune function, and thus helpful to the health of human bodies.

2. Description of the Prior Art

Nowadays most people live under nervousness, pressure of work, often short of exercise, easily getting fatigue, with blood circulation not so smooth. So they are apt to suffer sourness and pains to the muscles, and may buy pain patches to attach on sour or painful sections of the body, by at first peeling off a protective film. The medicinal paste may worsen owing to the temperature changes, in addition, the paste will not adhere closely on an elbow or knee with a curvature, lowing the treating function. If the paste is taken off, it is hardly reusable because its adhesiveness may be lost or bodily hairs may attach thereon, has to be discarded, making up some waste.

### SUMMARY OF THE INVENTION

The main purpose of the invention is to offer a healthful belt possessing good ventilating condition and far-infrared energy to supply medicating function for reinforcing blood circulation and metabolism, removing fatigue, lessening aching pains, with usability repeatable.

The feature of the invention is an elastic cloth and a silica gel layer deposited on an inner surface of the elastic cloth. The silica gel layer is provided with plural air holes and far-infrared energy for good ventilation and medicating effectiveness.

### BRIEF DESCRIPTION OF DRAWINGS

This invention will be better understood by referring to the accompanying drawings, wherein:

FIG. 1 is a perspective view of a healthful belt formed as a cylindrical shape in the present invention;

FIG. 2 is a partial magnified cross-sectional view of the healthful belt in the present invention;

FIG. 3 is a perspective view of the healthful belt in the present invention, showing it worn on an elbow and knees;

FIG. 4 is a perspective view of a second embodiment of a healthful belt formed rectangular in the present invention; and,

FIG. 5 is a perspective view of the healthful patches in the present invention, showing them practically used on a section of a human body.

### DETAILED DESCRIPTION OF THE PREFERRED EMBODIMENT

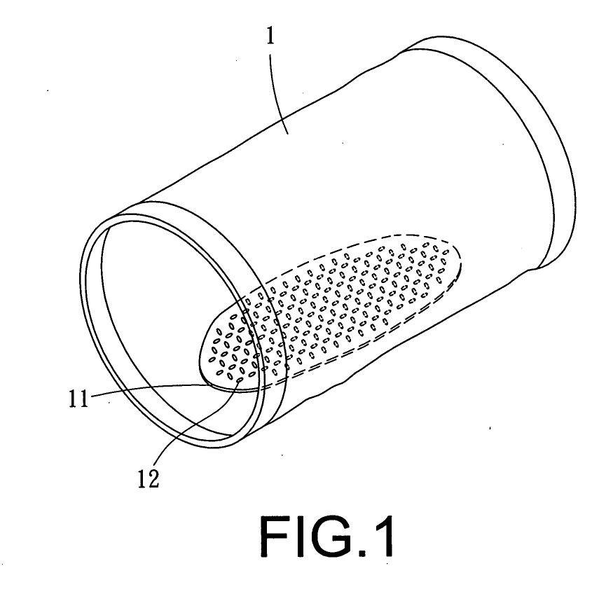

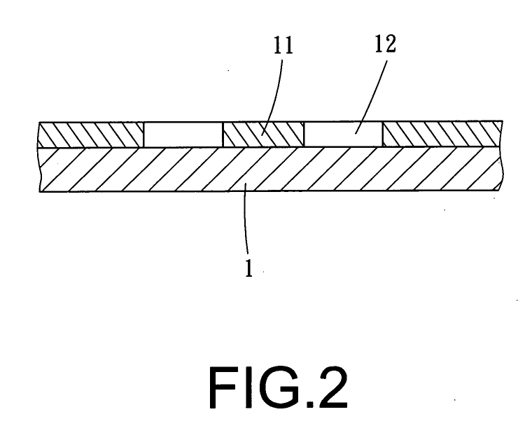

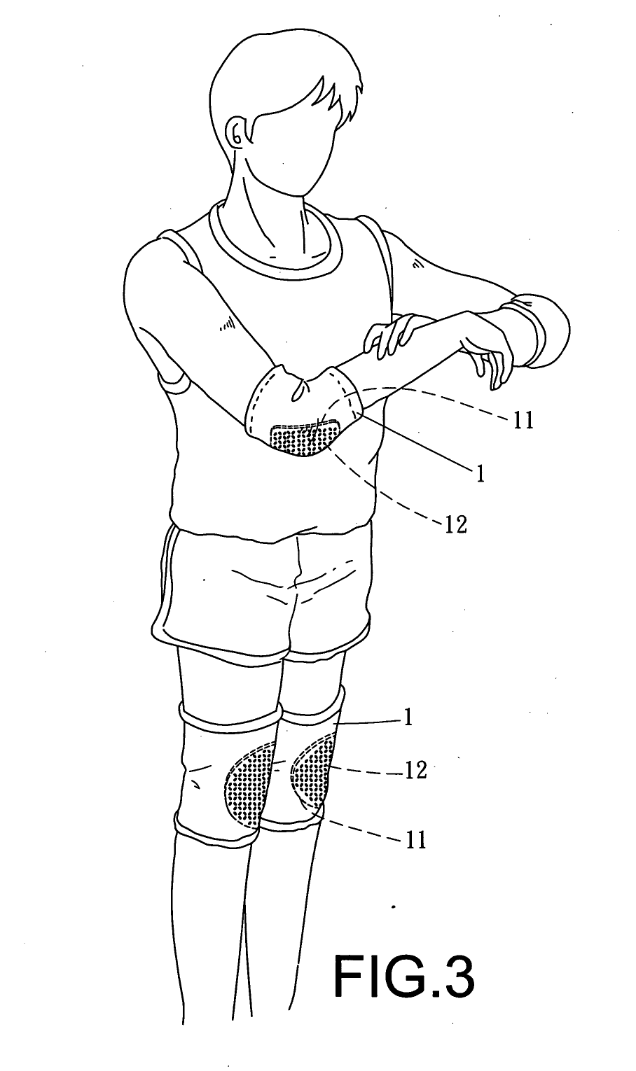

A preferred embodiment of a healthful belt 1 in the present invention, as shown in FIGS. 1 and 2, includes an elastic cloth of a cylindrical shape, and a silica gel layer 11 deposited on an inner surface of the elastic cloth and provided with plural air holes 12 for ventilation and far-infrared energy. Then the healthful belt is worn on a sour or painful section of a body, such as an elbow, a knee, etc. as shown in FIG. 3, letting the silica gel layer 11 directly contact the skin so that the far-infrared energy may sink into the skin organization and the joint, promoting blood circulation, healing fatigue, cutting down sourness and pains, reinforcing metabolism, enhancing immune function, and helpful to the health of human bodies.

Then the healthful belt is easy to use, possible to be washed repeatedly and thus helpful for protecting the environment. In addition, the silica gel layer 11 is soft and provided with the plural air holes 12 for good ventilation, so a person can feel comfortable when it contacts the skin of the person in using.

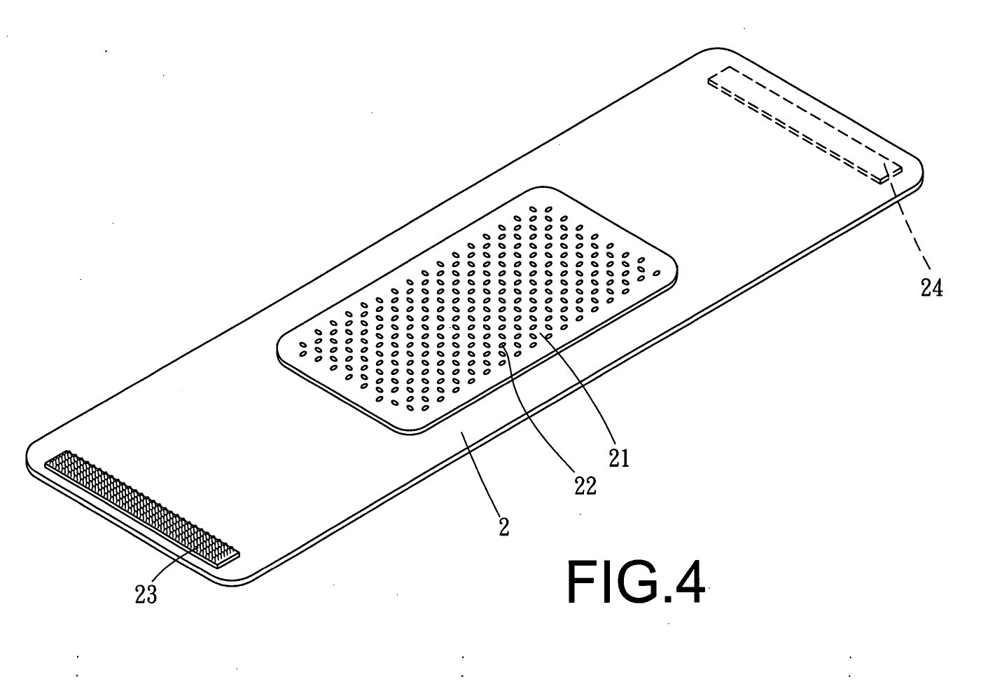

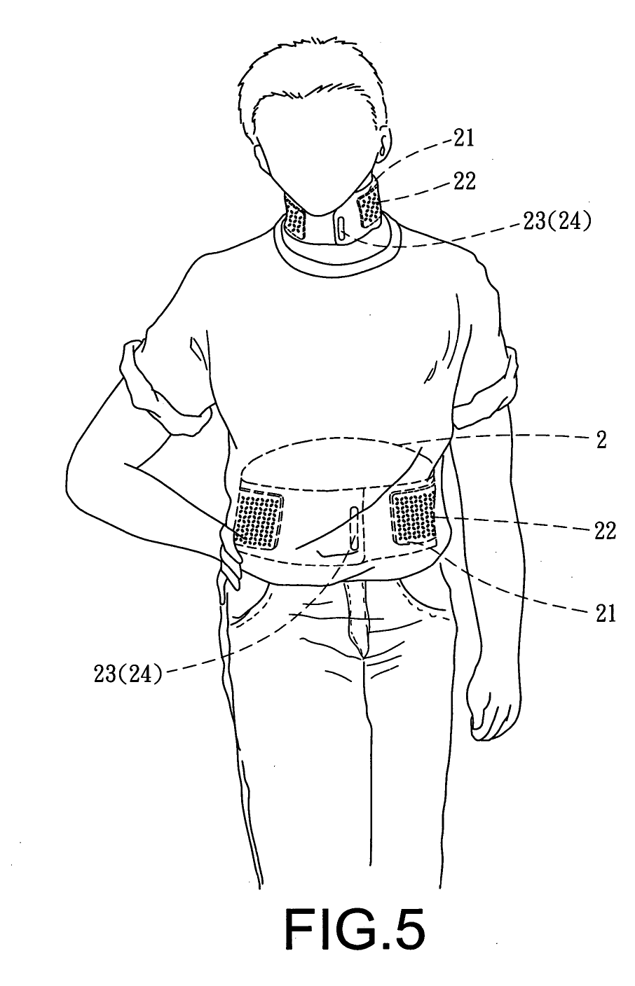

Next, FIG. 4 shows a second embodiment of a healthful belt 2, which is shaped rectangular, composed of an elastic cloth and a silica gel layer 21 deposited on the surface of the elastic cloth. The silica gel layer 21 is provided with plural air holes 22 for ventilation, and far-infrared energy for medicating effectiveness. Then a first Velcro member 23 is mounted on one side of the belt 2, and a second Velcro member 24 is mounted on the other side of the belt 2 for temporarily adhering with the first Velcro member 23 for stably positioning the belt 2 on a section of a body. Then as shown in FIG. 5, when the healthful belt 2 is worn around a painful section of a body, with the two Velcro members adhered together and with the silica gel layer 21 directly contacting the skin of a user, the far-infrared energy may sink into the skin organization and the joint in the same way described in the first embodiment. Then it can promote blood circulation, assuaging fatigue, reducing sourness and pains, reinforcing metabolism, enhancing immune function, and greatly helpful to the health of a body. Moreover, the healthful belt 2 can be taken off the body by separating the two Velcro members 23 and 24, convenient for washing, usable repeatedly, and helpful for protecting the environment.

The invention has the following advantages as can be seen from the foresaid description.

1. The far-infrared energy contained in the silica gel layer can promote blood circulation, lessening fatigue, reducing sourness and pains, reinforcing metabolism, upgrading immune function, and largely helpful to the health of a body.

2. It is easy to wash, usable repeatedly, helpful to the environment protection.

3. The silica gel layer is quite ventilating owing to the air holes and soft as well, so a person may feel comfortable in using the healthful belt.

While the preferred embodiment of the invention has been described above, it will be recognized and understood that various modifications may be made therein and the appended claims are intended to cover all such modifications that may fall within the spirit and scope of the invention.

What is claimed is:

1. A healthful belt comprising a cloth and a silica gel layer deposited on an inner surface of said cloth, said silica gel layer provided with plural air holes and far-infrared energy.

2. The healthful belt as claimed in claim 1, wherein said cloth is elastic cloth.

3. The healthful belt as claimed in claim 1, wherein said belt is shaped cylindrical.

4. The healthful belt as claimed in claim 1, wherein said belt is shaped rectangular.

5. The healthful belt as claimed in claim 4, wherein said healthful belt has a Velcro member respectively at two sides for mutually adhering temporarily to keep said healthful belt stably on a section of a body.
